(19) United States
(12) Patent Application Publication
(10) Pub. No.: US 2006/0153929 A1
(43) Pub. Date: Jul. 13, 2006

(54) Use of solid phase synthesis to modify and to assemble nanoparticles

(75) Inventor: Philip Leslie Drake, Taipei (TW)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Industrial Technology Research Institute

(21) Appl. No.: 11/034,109

(22) Filed: Jan. 11, 2005

(51) Int. Cl. H01L 21/00; A61K 33/00

(52) U.S. Cl. 424/600; 438/1; 977/900

(57) ABSTRACT

Methods of modifying nanoparticle surfaces using solid phase synthesis, methods of releasing nanoparticles from solid phase synthesis supports using photolytic cleavage, and methods of assembling nanoparticle structures are disclosed. Nanoparticles comprising a positionally distinguishable surface area comprising a ligand are also disclosed.

Provided herein are methods of modifying nanoparticle surfaces using solid phase synthesis, methods of releasing nanoparticles from solid phase synthesis supports using photolytic cleavage, and methods of assembling nanoparticle structures. Also provided are nanoparticles comprising a positionally distinguishable surface area comprising a ligand.

The composition, size, shape, and surface functionality can affect the material properties of nanoparticles. Individual nanoparticles can be assembled into ensembles or supramolecular structures comprising multiple nanoparticles and which can exhibit novel properties useful in devices such as electronic and optical devices. The properties of nanoparticle ensembles can depend on the ability to control the assembly of individual nanoparticles, for example into chains (one-dimensional), meshes (two-dimensional), or three-dimensional structures. To assemble complex nanoparticle structures it would be advantageous to develop the capability to modify a nanoparticle surface at positionally distinguishable surface areas and to assemble nanoparticles and/or subassemblies comprising more than one nanoparticle in a controllable and reproducible manner. As disclosed herein, this can be accomplished by applying solid phase synthesis methods to manipulate the functionalization of nanoparticles and to assemble nanoparticle structures while nanoparticles or subassemblies of nanoparticles are immobilized on a support.

Provided are methods of forming a nanoparticle surface comprising a second ligand coupled to the nanoparticle surface comprising, contacting a nanoparticle surface with a functionalized support surface comprising a first ligand, coupling the first ligand to the nanoparticle surface; and releasing the nanoparticle surface from the functionalized support surface to form a modified nanoparticle surface comprising the second ligand.

Also provided are methods of releasing a nanoparticle coupled to a surface comprising photolytically cleaving a photolabile linkage.

Also provided are nanoparticles comprising a surface formed by the foregoing methods.

Also provided are nanoparticles comprising a surface comprising a positionally distinguishable surface area, wherein the positionally distinguishable area comprises a ligand.

Also provided are methods of assembling a nanoparticle structure, comprising providing a functionalized support surface and a first subassembly, immobilizing the first subassembly to the functionalized support surface, and coupling a second subassembly to the first subassembly to form a nanoparticle structure immobilized on the functionalized support surface, wherein the first and second subassemblies comprise a nanoparticle chosen from the same nanoparticle and a different nanoparticle.

Also provided are nanoparticle structures formed by the foregoing methods of assembling nanoparticle structures.

Also provided are nanoparticle structures comprising a plurality of nanoparticles, wherein each of the plurality of nanoparticles comprises a positionally distinguishable surface area comprising a ligand.

These and other features of the present disclosure are set forth herein.

### DESCRIPTION OF THE DRAWINGS

The skilled artisan will understand that the drawings, described below, are for illustration purposes only. The drawings are not intended to limit the scope of the present teachings in any way.

FIGS. 1A-1C illustrate modification of a nanoparticle surface according to certain embodiments.

FIGS. 2A-2C illustrate modification of a ligand coupled to a nanoparticle surface according to certain embodiments.

FIG. 3 illustrates photolytic cleavage of a photolabile moiety of a ligand coupling a nanoparticle to a support surface according to certain embodiments.

FIG. 4 is a chart showing the absorbance spectrum of nanoparticle samples according to certain embodiments.

FIGS. 5A and 5B illustrate modification of a nanoparticle surface to provide a positionally distinguishable surface area according to certain embodiments.

FIGS. 6A-6C illustrate modification of a nanoparticle surface according to certain embodiments.

FIG. 7 illustrates modification of a nanoparticle surface according to certain embodiments.

FIGS. 8A-8C illustrate assembly of a one-dimensional nanoparticle structure according to certain embodiments.

FIG. 8D illustrates assembly of a two-dimensional nanoparticle structure according to certain embodiments.

FIGS. 9A-9B are charts showing the intensity of the surface plasmon resonance peak of nanoparticles in solution according to certain embodiments.

FIGS. 10A-10D illustrate assembly of a three-dimensional nanoparticle structure according to certain embodiments.

Unless otherwise indicated, all numbers expressing dimensions, tolerances, and so forth used in the specification and claims are to be understood as being modified in all instances by the term “about”. Accordingly, unless indicated to the contrary, the numerical parameters set forth herein are approximations that may vary depending upon the desired properties to be obtained.

In this application, the use of the singular includes the plural unless specifically stated otherwise. In this application, the use of “or” means “and/or” unless stated otherwise. Furthermore, the use of the term “including”, as well as other forms, such as “includes” and “included,” is not limiting. Also, terms such as “element” or “component” encompass both elements and components comprising one unit and elements and components that comprise more than one subunit unless specifically stated otherwise.

In accordance with the usual meaning of “a” and “the” in patents, reference, for example to “a” ligand or “the” ligand is inclusive of one or more ligands.

The section headings used herein are for organizational purposes only, and are not to be construed as limiting the subject matter described.

Description of Various Embodiments

Nanoparticle surfaces can be modified using solid phase synthesis methods. Solid phase synthesis (SPS) refers to a methodology in which chemical reactions and transformations can be carried out on a surface of a support and/or on a material such as a molecule coupled to a support surface. By applying SPS methods to nanoparticle surface modification it is possible to perform controlled ligand exchange reactions at a subset of ligands coupled to a nanoparticle surface and to perform controlled ligand reactions at positionally distinguishable areas of a nanoparticle surface.

In SPS modification of nanoparticle surfaces, a support surface can be functionalized with ligands using well known SPS coupling reactions. Nanoparticles can then be contacted to the functionalized support surface. A nanoparticle surface can, for example, be functionalized with a second set of ligands. Ligands coupled to the support surface and to the nanoparticle surface can then react to couple the nanoparticle to the support surface via the reacted ligands. Further reactions can be applied to the nanoparticle immobilized on the support surface. For example, ligands can be coupled to an immobilized nanoparticle and/or a subassembly comprising one or more nanoparticles can be coupled to the immobilized nanoparticle. Following controlled manipulation of the immobilized nanoparticle, the coupling ligand can be cleaved to release the nanoparticle from the support surface. This can result in the nanoparticle being modified only at the ligand or group of ligands which were coupled to the support surface. When coupled to the support surface, the nanoparticle can also be modified on a surface area other than that where the nanoparticle is coupled. For example, a ligand can be coupled to the nanoparticle surface at an area other than the area coupled to the support surface. This can provide for modification of the nanoparticle surface at positionally distinguishable areas.

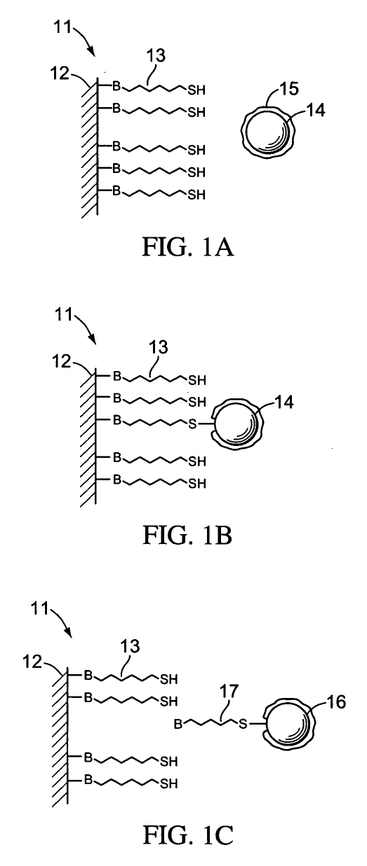

A method of modifying a surface of a nanoparticle is shown in FIGS. 1A-1C. A functionalized support surface 11 includes a support 12 and ligands 13 coupled to support 12. Ligands 13 can include functionality such as —SH groups, amines, substituted amines, carboxylic acids, esters, aldehydes, ketones, halogen groups, alcohols, amides, and the like, capable of reacting with other functional moieties such as terminal moieties of ligands coupled to a nanoparticle surface and/or capable of reacting directly with a nanoparticle surface. Ligands 13 can be coupled to support 12 by a releasable bond to group B, or by a linkage comprising a chemical entity, such as for example, a ligand. In a first step, nanoparticles 14 can be contacted with functionalized support surface 11 (FIG. 1A). Nanoparticles 14 can be stabilized with a compound such as a surfactant 15 and/or a ligand (not shown). In a second step, the surface of nanoparticle 14, such as a gold (Au) nanoparticle, can react with ligands 13, coupling nanoparticle 14 to functionalized support surface 11 (FIG. 1B). FIG. 1B shows nanoparticle 14 immobilized on support 12 via a single ligand 13. In a third step, ligand 13 can be cleaved to release nanoparticle 14 from functionalized support surface 11 (FIG. 1C). A modified nanoparticle 16 includes a second ligand 17 coupled to the nanoparticle surface.

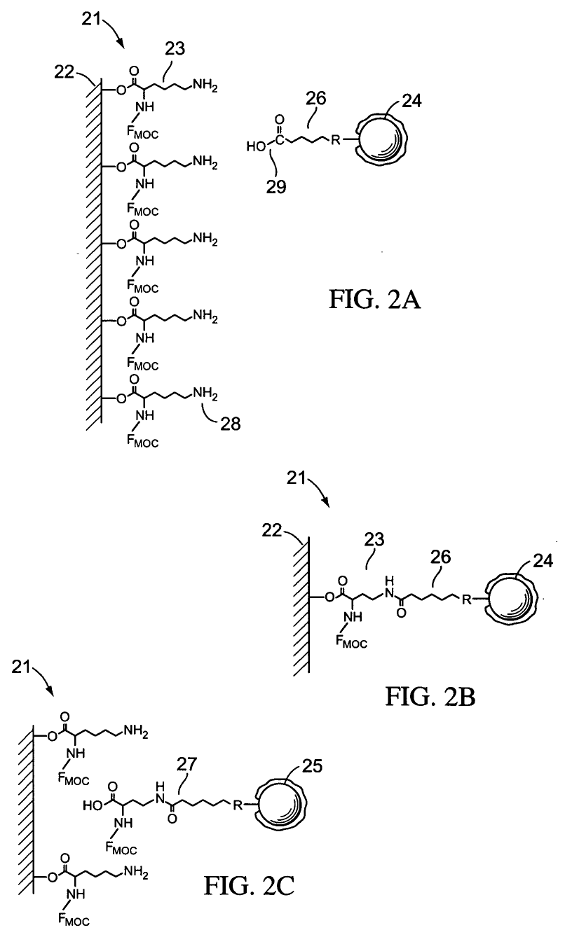

As shown in FIGS. 2A-2C, in certain embodiments, a nanoparticle 24 can comprise a ligand 26 comprising a functional terminal group or a terminal group capable of being functionalized 29. Ligand 26 can be coupled to nanoparticle surface 24 by SPS or solution phase methods. In FIGS. 2A-2C, ligands 26 and 27 are shown coupled to nanoparticle surface 24 by group R which can be a bond or chemical entity and can couple ligands 26 and 27 by covalent or non-covalent forces. A nanoparticle comprising ligand 26 can be contacted with a functionalized support surface 21 comprising first ligands 23 coupled to a support 22 and having a functional terminal group or a terminal group capable of being functionalized 28 (FIG. 2A). Terminal groups 28 and 29 can be capable of reacting with each other. When contacted, terminal groups 28 and 29 can react to couple nanoparticle 24 to support surface 22 via ligands 23//26 (FIG. 2B). Following optional further manipulation of immobilized nanoparticle 24, nanoparticle 24 can be released from functionalized support surface 21, to provide a modified nanoparticle 25 that includes a third ligand 27 coupled to the nanoparticle surface (FIG. 2C).

Functionality, functional, or functionalized refers to a group of atoms or chemical moieties that provides an intended utility. The utility can be a chemical property or a physical property. Examples of chemical properties include general reactivity, specific chemical reactivity, photochemical activity, protection, solubility, and/or ionic. Examples of physical properties include electronic, steric, optical, and/or magnetic. Functional moieties can be located at the terminal ends of a ligand, and/or incorporated into the backbone of a ligand. Examples of functional terminal groups include thiol, —SH, groups used for bonding to Au surfaces, and —COOH or amino groups for bonding to polymeric support surfaces. Examples of protecting groups include, for example, Fmoc (9-Fluorenylmethoxycarbonyl), BOC (t-Butoxycarbonyl), Cbz (Carbobenzyloxy), Z (Benzyloxycarbonyl), Dde (1-(4,4-dimethyl-2,6-dioxocyclohexylidene)ethyl), Trityl (triphenylmethyl), Ac (Acetyl), and Bz (Benzoyl). Examples of functional groups incorporated into the backbone of a ligand include photolabile moieties such as 2-Bromopropiophenone groups, o-Nitrobenzyl groups such as 3-Hydroxymethyl-4-nitrophenoxymethyl, and alkoxy substituted o-Nitrobenzyl groups such as N-(1,5-Methoxy-2-nitro-oxyphenyl)-ethyl acetamide, and chemically cleavable moieties such as moieties capable of being cleaved via acid catalyzed reactions such as lysine, Wang linkers, RINK linkers, KNORR linkers, benzyl alcohol linkers, chlorotrityl linkers, and BOC protecting groups, and via base catalyzed reactions such as HMBA-MBHA (4-Hydroxymethylbenzoic acid-4-methyl-benzhydrylamine) linkers, haloalkane linkers such as TentaGel S Br, hydroxyalkane linkers such as TentaGel S OH, or the Fmoc protecting group. Functional groups can be protected with a chemical moiety capable of being removed during chemical processing steps.

Nanoparticles are variously defined in the art as materials comprising less than about 1 million atoms, or materials exhibiting a diameter less than about 1,000 nanometers. In certain embodiments, nanoparticles can exhibit diameters less than about 100 nanometers. Nanoparticles can generally be spherical, triangular, rod-like, cubical, vertex-truncated cubical, as well as other shapes, and/or mixtures thereof. Nanoparticles can comprise a single material or multiple materials. For example, nanoparticles can comprise metals such as Au, Ag, Pt, Ti, Al, Si, Ge, Cu, Cr, W, Fe, Pd, and/or Ir, metal oxides such as metal oxides of any of the foregoing, semiconductors such as group III-V and group II-VI semiconductors such as CdSe, CdS, CdTe, and/or GaAs, organic materials such as polystyrene, HMP-PEGA polyacrylamide, polyacrylic acid, PEG (Polyethylene glycol), and/or PLGA (Poly(lactic-co-glycolic acid), and/or composites of any of the foregoing. Nanoparticles can be formed by methods known in the art. For example, Au nanoparticles can be synthesized using a two-phase (water/toluene) method, as described, for example, by Brust, M., et al., J. Chem. Soc., Chem. Commun., 1994, p. 801-802, to produce Au nanoparticles exhibiting a diameter ranging from 2 nm to 9 nm depending, for example, upon the surfactant or ligand employed.

Nanoparticles useful in methods of the present disclosure can comprise a treated or untreated surface. However, since untreated nanoparticles can agglomerate, flocculate, and/or precipitate, individual nanoparticles are typically stabilized by treating the nanoparticle surface with a compound such as a surfactant or ligand which can reduce or eliminate ionic, electrostatic and/or other inter-nanoparticle interactions between nanoparticles. Thus, nanoparticle surfaces can comprise the same materials as the bulk of the nanoparticle or can comprise a thin film, coating, multilayer, and/or monolayer of a material that is different than that of the bulk.

In certain embodiments, a multilayer or monolayer disposed on a nanoparticle surface can comprise ligands coupled to the nanoparticle surface, and in some embodiments, ligands can be covalently or non-covalently coupled to a nanoparticle surface. Although a nanoparticle can comprise a single ligand, it can also be useful to cover a nanoparticle surface with a plurality of ligands.

In certain embodiments, nanoparticle surfaces useful in methods of the present disclosure can comprise a coating of one or more surfactant. A surfactant can adhere to a nanoparticle surface by covalent or non-covalent forces. Non-covalent force refers to the interaction of a compound and another compound or material wherein a covalent bond is not formed between the compound and the other compound or material. Examples of non-covalent bonding include, van der Waals interactions, hydrogen bonding, and electrostatic interactions (also known as ionic bonding). Examples of useful surfactants include Tetraoctylammonium bromide (TOABr), Cetyltrimethylammoniumbromide (CTAB), Sodium dodecylsulfate (SDS), stearic acid and sodium stearate, and/or thioalkanes.

Ligands can be any material capable of reacting with a nanoparticle surface and/or a support surface to form a linkage that is sufficiently stable to subsequent manipulation. Thus, in certain embodiments, a ligand can be any polyatomic chemical entity that is or becomes coupled to either a nanoparticle surface and/or a support surface. In certain embodiments, a ligand can be covalently coupled to a surface. A ligand can comprise at least one functional group capable of covalently bonding to a surface such as a nanoparticle and/or support surface, and/or at least one functional group capable of reacting, and/or capable of being modified to react. Examples of ligands include biopolymers such as antibodies, proteins, biomolecules, amino acids, peptides, DNA, and RNA, and other organic polymers such as surfactants, synthetic polymers, polysaccharides, lipids, fatty acids, and the like.

A ligand can be a multi-dentate ligand comprising multiple branches with the same or multiple functionalities. For example, a multi-dentate ligand can comprise multiple branches with certain branches comprising a terminal functionality capable of binding to a surface. Other branches of a multi-dentate ligand can comprise functionalities capable of bonding to other ligands or capable of being modified to provide additional functionality. Certain branches can also include chemically inert functionalities that represent terminal blocking groups or elements that impart steric features.

A ligand can be, in whole or in part, linear or non-linear, rigid or flexible, and can comprise functional groups oriented in different directions with respect to other functional groups. Such wide diversity in ligand properties can be used to produce a variety of controlled supramolecular nanoparticle structures.

A support surface can be any appropriate material. The material of a support surface can be selected to be insoluble in the solvent used in SPS reactions, can facilitate binding of particular ligands, and/or can exhibit desirable properties such as optical transparency, and/or electrical conductivity either in whole or in part. A support surface can have any appropriate shape or form. For example, a support surface can be in the form of a bead, a planar substrate, an essentially planar substrate with topographical features, an inner wall of a cylinder, or an outer wall of a cylinder. A support surface can comprise a single material or more than one material. For example, a support surface comprising more than one material can include layers or thin films of polymers disposed on a solid support comprising a metal, a metal oxide, a semiconductor such as silicon, polymer, silica, glass, and the like. Examples of useful polymers which can be used either as a solid support or layer include polystyrene, polyethyleneglycol, polyacrylamides, polysiloxanes, silica, and/or dendrimers. Examples of commercially available resin beads include JandJel resins, HMPA-PEGA resins, Wang resins, Merrifield resins, TentaGel resins, PAM (Phenylacetoamidomethyl) resins, and/or REM (4-(Benzyloxy)benzyl acrylate) resins.

In certain embodiments, supports can be insoluble in solvents used in methods of the present disclosure. In certain embodiments, supports may or may not change dimensions when exposed to solvents useful in methods of the present disclosure. Dimensional changes of a support surface can be useful in controlling the surface density of ligands coupled to a support surface.

A ligand can be bound to a support surface to provide a functionalized support surface. Chemistries for functionalizing support surfaces are well known in the art, and functionalized support surfaces are commercially available. Ligands can be coupled to a support surface via a linkage that is not intended to be cleavable during SPS processing. In certain embodiments, ligands can be coupled to a support surface via a moiety that can be cleaved by acid catalyzed reactions or by base catalyzed reactions. In certain embodiments, ligands can be coupled to a support surface by a photolytically cleavable moiety. An example of a photolytically cleavable moiety is a photolabile system using a o-Nitrobenzyl intermolecular redox reaction. Such a system can be provided by coupling the amino acid cysteine (Cys) to a resin through amide bond formation. A photolabile cysteine linkage can be prepared by reacting an SPS resin with an initial loading of —NH₂ groups, characterized, for example by a density of less than about 1 mmol-g⁻¹ with the carboxylic acid group of cysteine using Benzotriazol-1-yl-oxytripyrrolidino-phosphonium hexafluorophosphate (PyBOP) conditions to yield a cysteine group coupled to the support surface. A cysteine group can be a terminal cysteine group of a ligand.

Ligands immobilized on a support surface can be coupled to a nanoparticle surface using the same or different chemistries as used to couple a ligand to a support surface. Such methods are well known in the art. The free, uncoupled end of an immobilized ligand can be coupled directly to a nanoparticle surface, or to a material covalently or non-covalently coupled to the nanoparticle surface. For example, the free end of a ligand can react with a terminal functional group of a ligand covalently coupled to a nanoparticle surface. In certain embodiments, wherein the nanoparticle is an Au nanoparticle, ligands having terminal thiol groups can covalently react with the Au nanoparticle surface. Terminal thiol groups can displace materials such as surfactants and/or thiolate protecting groups to covalently couple Au nanoparticles to an SPS support surface. Coupling of Au nanoparticles to an SPS support surface can be monitored, for example, by observing the color change of the Au nanoparticle solution. Isolated solution-phase Au nanoparticles can be characterized by a surface plasmon resonance (SPR) absorption at a wavelength of 520 nm. When Au nanoparticles are contacted with an SPS support surface comprising an activated Cys-SH terminated ligand, the amplitude of the SPR in the solution phase diminishes. A decrease in the amplitude of the SPR absorption is indicative of Au nanoparticle binding to the SPS support surface.

Following coupling of nanoparticles to an SPS support surface, the system can be washed. For a system comprising thiol-terminated ligands coupled to Au nanoparticles, toluene, dichloromethane, acetone, hexane, hydrophobic solvents, and the like, are examples of appropriate washing solutions. During washing, the system can optionally be sonicated and/or agitated. Washing can be used to remove Au nanoparticles that are not coupled to the SPS support surface. In certain embodiments, repeated washing cycles can be used. Following washing, a solvent and any additional reactants can be added to the system appropriate for carrying out the decoupling reaction to release the nanoparticles from the SPS support surface. In certain embodiments, nanoparticles can be stabilized prior to washing. For example, nanoparticles can be stabilized with TOABr before washing with dodecanethiol.

The cleavage chemistry and/or mechanism appropriate for particular ligands can be selected. In certain embodiments, acid catalyzed reactions or based catalyzed reactions can be used. An example of these systems include the acid catalyzed cleavage of Fmoc-Lys(Dde) ligands in a solution of 60% Trifluoroacetic acid (TFA) in an inert solvent such as DMF (N,N-Dimethylformamide) (60% TFA, 2.5% TIS, 2.5% H₂O, 35% DMF, 24 h), or CH₂Cl₂ in which lysine is bonded to the nanoparticle. In this system the ligand is cleaved at the “ester-amide” group where the lysine is coupled to the surface. Acid catalyzed reactions can also be used to cleave ligands coupled to surfaces by carboxyl groups. Release protocols using acid catalyzed reactions or base catalyzed reactions typically require multiple steps of solvent exchange and removal to increase the nanoparticle yield and to remove residual acid which can affect subsequent nanoparticle coupling reactions.

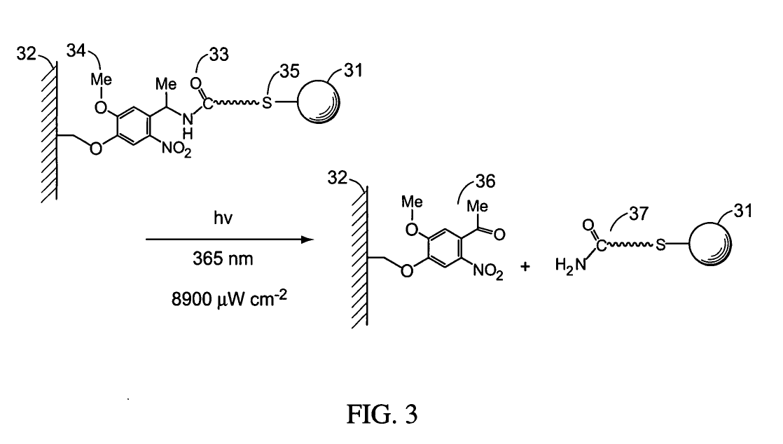

In embodiments wherein a ligand that couples a nanoparticle to a support surface incorporates a photolabile linkage, the nanoparticle can be released photolytically. Examples of useful photolabile moieties include 2-Bromopropiophenone groups, o-Nitrobenzyl groups such as 3-Hydroxymethyl-4-nitrophenoxymethyl, and alkoxy substituted o-Nitrobenzyl groups such as N-(1,5-Methoxy-2-nitro-oxyphenyl)-ethyl acetamide. FIG. 3 shows an example of a method of photolytically releasing a nanoparticle coupled to a support surface. FIG. 3 shows an Au nanoparticle 31 coupled to an SPS support 32 via a ligand 33 comprising a photolabile moiety 34. Au nanoparticle 31 is coupled to ligand 33 via a sulfide bond 35. The opposite end of ligand 33 is coupled to SPS support 32 via moiety 34. Irradiation of moiety 34 with UV radiation can induce photolytic cleavage of the photolabile linkage to produce a benzaldehyde derivative 36 coupled to SPS support 32 and free the amide-terminated ligand 37 to release Au nanoparticle 31. Any appropriate wavelength and power of the optical radiation can be used sufficient to induce the photochemical cleavage reaction. The appropriate wavelength and power can in part depend on the absorption profile and cross-section of a particular photolabile linkage, as well as the concentration and/or density of the reactants. For example, cysteine as well as many complex organic molecules exhibit significant absorption in the UV at wavelengths less than about 350 nm. In certain embodiments, appropriate power densities can range from 1 μW-cm⁻² to 10,000 μW-cm⁻², and in certain embodiments, can range from 100 mW-cm⁻² to 10,000 mW-cm⁻²

Photolabile moieties can be incorporated at any point in the ligand including as terminal groups or in the ligand backbone. FIG. 3 shows a photolabile moiety coupled to an SPS support surface and coupled to a nanoparticle via a photolabile linkage. In certain embodiments, a photolabile moiety can be coupled to a nanoparticle surface, and in certain embodiments, a photolabile moiety can be incorporated within the ligand backbone. A ligand can comprise more than one photolabile moiety wherein the more than one photolabile moiety is the same or different chemical species. In certain embodiments, a photolabile moiety can be deactivated or rendered photolytically inactive by a protecting group that can subsequently be removed to render the photolabile moiety photolytically active.

At least one advantage of methods of releasing nanoparticles from an SPS support surface by photolytic cleavage is that ligand-coupled nanoparticles can be directly released into a solvent suitable for use in subsequent reactions without the need to repeatedly wash, isolate and stabilize the released nanoparticles as necessary when acid- or base-catalyzed release protocols are employed.

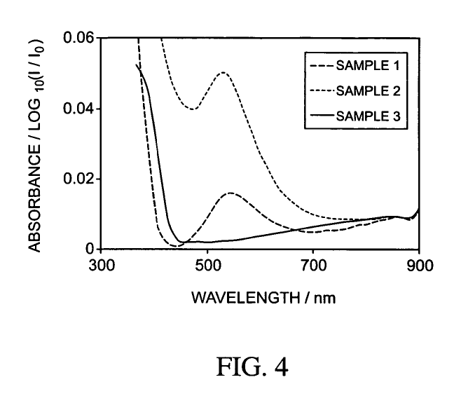

The photolytic-induced release of ligand-modified nanoparticles can be determined by monitoring the UV-Vis spectrum, and in particular the SPR region of the nanoparticle spectrum. Nanoparticles coupled to an SPS support surface via a ligand incorporating a cysteine moiety linked through a peptide bond to an o-Nitrobenzyl photolabile group in toluene were irradiated with UV light at a wavelength of 350 nm to induce the photochemical cleavage of the C—N bond of the cysteine moiety, thereby releasing the nanoparticle into a solvent such as toluene. As shown in FIG. 4, Sample 1, upon exposure to UV light the characteristic SPR peak associated with isolated Au nanoparticles forms in the toluene solution. The SPR peak appears in the wavelength range from about 500 nm to 600 nm. The SPR peak is absent in a UV irradiated sample containing only cysteine-coupled ligand bound to an SPS resin with no Au nanoparticles (FIG. 4, Sample 3). The UV-Vis spectrum of FIG. 4, Sample 2 corresponds to the spectrum obtained following UV irradiation of a sample comprising a lower mass ratio of Au nanoparticle to SPS support resin than in Sample 1. The magnitude of the SPR peak of Samples 1 and 2 quantitatively match the mass ratios of the respective samples. The UV-Vis spectrum of these solutions showed no change over the course of seven days indicating that the Au nanoparticle toluene solution was stable and free from aggregation, flocculation, and precipitation. Following photochemical decoupling the SPS support resins can be recovered by filtration, washed with fresh toluene, and dried under vacuum. The mass of the recovered resin can also be measured, and when using the methods disclosed herein, can be equivalent to that of the initial resin showing that all or nearly all nanoparticles coupled to the support surface can be released by the photolytic decoupling method.

The number and/or density of ligands coupled to a nanoparticle surface using SPS methods can in part be determined and controlled by the density of ligands coupled to a support surface and/or the density of ligands coupled to a nanoparticle surface. For example, when the density of ligands coupled to a support surface is sufficiently low with respect to the diameter of a nanoparticle, only a single ligand will become coupled to the nanoparticle. When the nanoparticle is released from the support surface, only a single ligand will be coupled to the nanoparticle. At higher support surface ligand densities, more than one ligand can be coupled to a nanoparticle, which upon release of the nanoparticle can produce more than one ligand coupled to the nanoparticle surface.

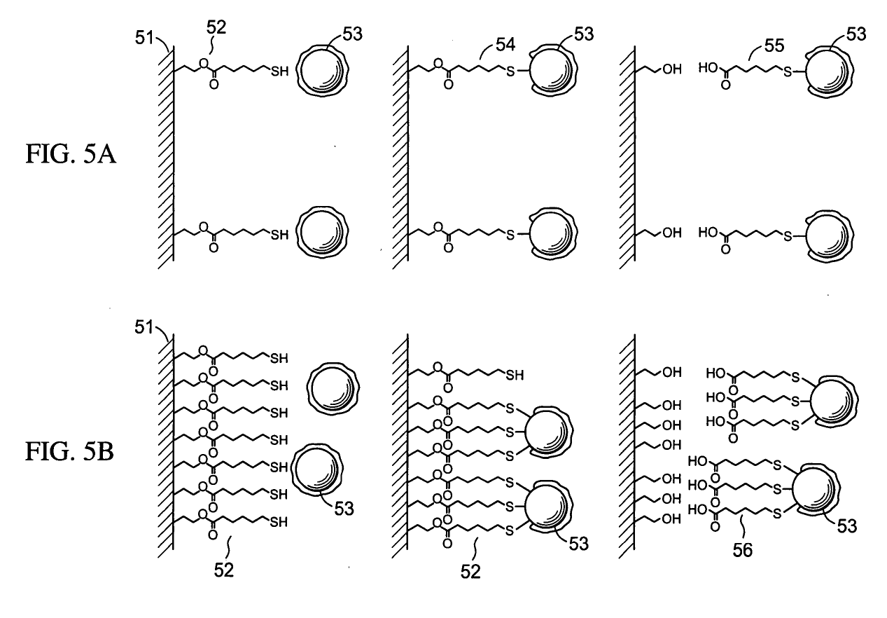

Examples of these embodiments are shown in FIGS. 5A and 5B. FIG. 5A shows a support surface 51 comprising a low surface density of ligands 52 with respect to the diameter of a nanoparticle 53. Following reaction, a single ligand 54 is coupled to nanoparticle 53, and upon release, nanoparticle 53 comprises a single ligand 55. An embodiment wherein multiple ligands are coupled to a nanoparticle surface is shown in FIG. 5B. FIG. 5B shows support surface 51 having a high surface density of ligands 52, such that multiple ligands 52 can couple to nanoparticle surface 53. Following release, nanoparticle 53 comprises a plurality of ligands 56.

Referring to FIG. 5B, plurality of ligands 56 coupled to nanoparticle 53 are disposed in a positionally distinguishable area which is in part determined by the surface density of ligands coupled to the support surface with respect to the diameter of the nanoparticle. Having ligands coupled to positionally distinguishable areas of a nanoparticle surface can be useful in assembling supramolecular structures of nanoparticles.

The surface density of ligands coupled to a support surface can, in part, be determined by the concentration of reactive ligands in the solution used to functionalize the surface, the density of active and/or reactive groups on the support surface prior to functionalization, and/or the conditions of the ligand coupling reaction. In certain embodiments, the density of ligands coupled to a surface can be controlled by activating specific sites on the support surface using, for example, lithographic, masking, and/or other methods used to define nano-scale features in the electronics industry. Once prepared, ligands can optionally be coupled to the activated sites to functionalize the surface.

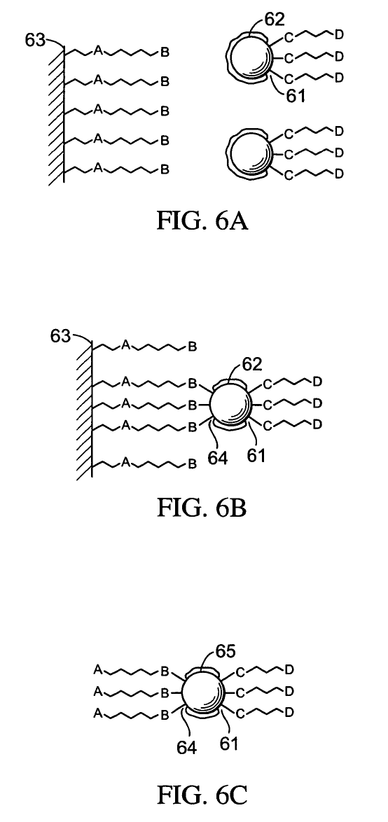

In certain embodiments, methods of the present disclosure can be used to modify and/or form nanoparticles comprising at least one positionally distinguishable area. Following the formation of a first positionally distinguishable area on a nanoparticle surface by any of the methods disclosed herein, additional positionally distinguishable areas can be formed by repeating the same and/or similar methods. FIGS. 6A-6C show steps used to form a nanoparticle surface comprising two positionally distinguishable areas. A first positionally distinguishable area 61 comprising ligand C-D is formed on a nanoparticle surface 62 using methods disclosed herein. Nanoparticle 62 can then be contacted with a support surface 63 comprising ligands A-B that are not reactive with ligands C-D within first positionally distinguishable surface area 61 (FIG. 6A). A second positionally distinguishable area 64 of nanoparticle surface 62 can then be coupled to support surface ligands A-B (FIG. 6B). First positionally distinguishable surface area 61 can be oriented away from support surface 63, by steric, electronic, ionic, and/or solubility effects. The surface of nanoparticle 62 can be coupled to a single ligand A-B or to a plurality of ligands A-B. Upon release from support surface 63, the nanoparticle comprises a first positionally distinguishable surface area 61 comprising ligands C-D, and second positionally distinguishable surface area 64 comprising ligands A-B (FIG. 6C). Ligands A-B and ligands C-D can be the same or different ligands, moieties B and C can be the same or different moiety, and moieties A and D can be the same or different moiety. Moieties A and/or B can be, for example, a photolabile moiety. Other surface areas 65 of the nanoparticle can not be functionalized and can comprise, for example, non-functional ligands or a surfactant. Repetition of the general steps shown in FIG. 6 can produce additional positionally distinguishable surface areas on a nanoparticle.

More than one positionally distinguishable surface area can comprise the same differentiable ligand, or different differentiable ligands. For example, a first positionally distinguishable area can comprise a first ligand having a first functionality. A second positionally distinguishable area can comprise the first ligand having a second functionality, a second ligand having the first functionality, or a second ligand having a second functionality. The second functionality can comprise, for example, a protecting group attached to a moiety having the first functionality, or can be a different chemical moiety. Nanoparticles having more than one positionally distinguishable area comprising ligands with different functionalities can be used with sequential reaction chemistries for the controlled assembly of supramolecular nanoparticle structures.

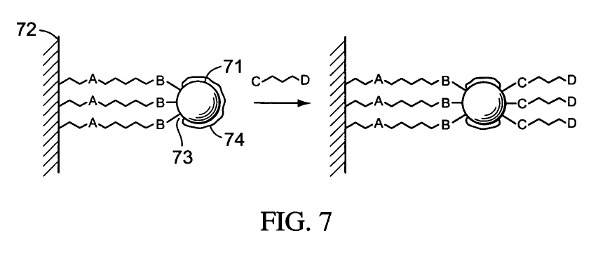

SPS can further be used to control the functionalization of immobilized nanoparticle surfaces. FIG. 7 shows a nanoparticle 71 coupled to a support surface 72 via a plurality of ligands A-B of a positionally distinguishable surface area 73. In certain embodiments, the nanoparticle surface 74 away from support surface 72 can be modified by exposing nanoparticle surface 74 to a solution comprising a ligand C-D. Ligands C-D can be reacted and coupled to nanoparticle surface 74. While remaining immobilized on support surface 72, nanoparticle 71 comprising ligands C-D can then be washed and/or exposed to further processing. Nanoparticle 71 comprising ligands A-B and C-D wherein each set of ligands is disposed in a separate positionally distinguishable area of nanoparticle 71 can subsequently be released from the support surface using the methods disclosed herein. Each process can be repeated one or more times while nanoparticle 71 is immobilized. Furthermore, the nanoparticle surface and/or ligands coupled to the nanoparticle surface can be further modified subsequent to one or more previous modification processes. For example, a nanoparticle can be released from a first support surface, coupled to ligands disposed on a second support surface, and released to form a nanoparticle having two sets of ligands in two positionally distinguishable areas where the ligands can be the same or different. Thus, SPS can be used as a tool to modify a nanoparticle surface where the steps of modification, washing, and purification can be carried out while the nanoparticle is immobilized on the support surface. Additional ligand binding reactions can be employed to form additional positionally distinguishable areas while the nanoparticle is coupled to the SPS support surface, and/or a nanoparticle characterized by one or more positionally distinguishable surface areas can be coupled to an SPS support surface and subsequently modified.

A modified nanoparticle surface comprising a ligand can be further modified after the nanoparticle is released from the support. For example, in certain embodiments, the released, modified nanoparticle can be reacted with additional ligands and/or nanoparticles in solution. In certain embodiments, the released, modified nanoparticle can be coupled to another functional support surface and further modified using methods disclosed herein.

SPS methods can be used to modify functional ligands coupled to a nanoparticle as well as to modify protected surface areas. For example, a ligand coupled to a nanoparticle comprising a terminal functional group reactive with a terminal functional group of a ligand coupled to an SPS support surface can be reacted to couple the nanoparticle to the SPS support surface via a composite ligand comprising a first and second ligand. The composite ligand can then be cleaved to produce a new ligand coupled to the nanoparticle exhibiting, for example, a different functionality and/or property.

Such SPS manipulation of nanoparticle surfaces can be performed in a batch process where each step is discontinuous, or in a continuous process such as a flow through system, such as using a microfluidic device, capillary, or column. SPS manipulation of nanoparticles can also include multiple processes where the nanoparticle surface is modified while immobilized on an SPS support and/or modified in solution.

Functionalized nanoparticles modified or formed by the methods disclosed herein can be used to construct supramolecular structures comprising more than one nanoparticle. Nanoparticle structures refer to structures or assemblies of more than one nanoparticle wherein adjacent nanoparticles are held together by covalent and/or non-covalent forces. In certain embodiments, adjacent nanoparticles can be held together by covalent bonding, such as via at least one ligand covalently bound to each of the coupled nanoparticle surfaces. Functionalized nanoparticles can be assembled using well known coupling chemistries.

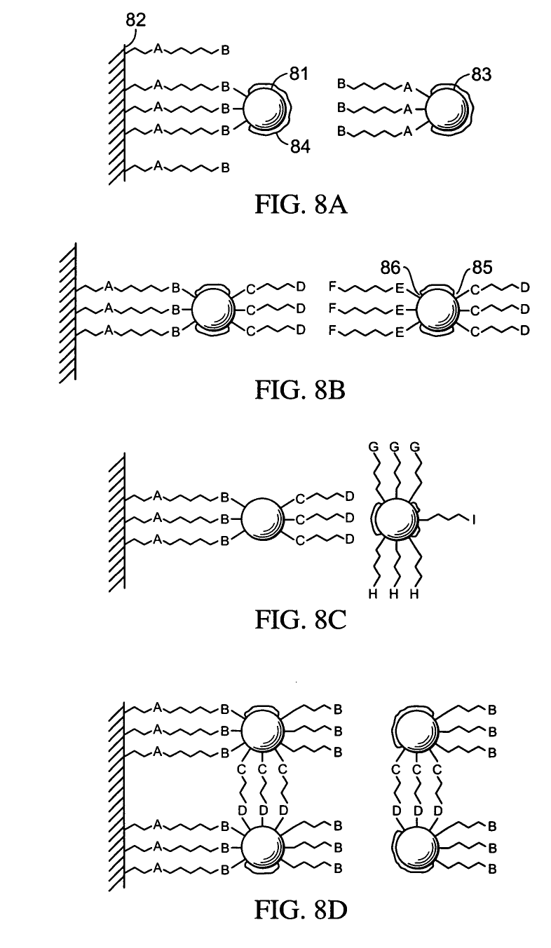

FIGS. 8A-8D show examples of methods of assembling nanoparticle structures using sequential chemistry and nanoparticles having at least one positionally distinguishable surface area. FIG. 8A shows a nanoparticle 81 immobilized on an SPS support surface via ligands A-B. While immobilized, nanoparticle surface 84 can react with solution phase molecules such as ligands, and/or functionalized nanoparticles such as nanoparticle 83 comprising ligands A-B. Using known SPS methods, linear chains of nanoparticles can be assembled in a sequential process while the growing chain or nanowire remains immobilized on the SPS support. Upon completion of the process, the nanowire can be released from the SPS support to provide a nanowire comprising a controlled sequence of individual nanoparticles and ligands. As shown in FIG. 8B, a linear chain of nanoparticles can be assembled by coupling nanoparticles comprising two positionally distinguishable areas 85 and 86, which each area comprising at least one ligand E-F and C-D, respectively. Branched and two-dimensional networks of nanoparticles can be produced by reacting nanoparticles comprising more than two functionalized areas (FIG. 8C).

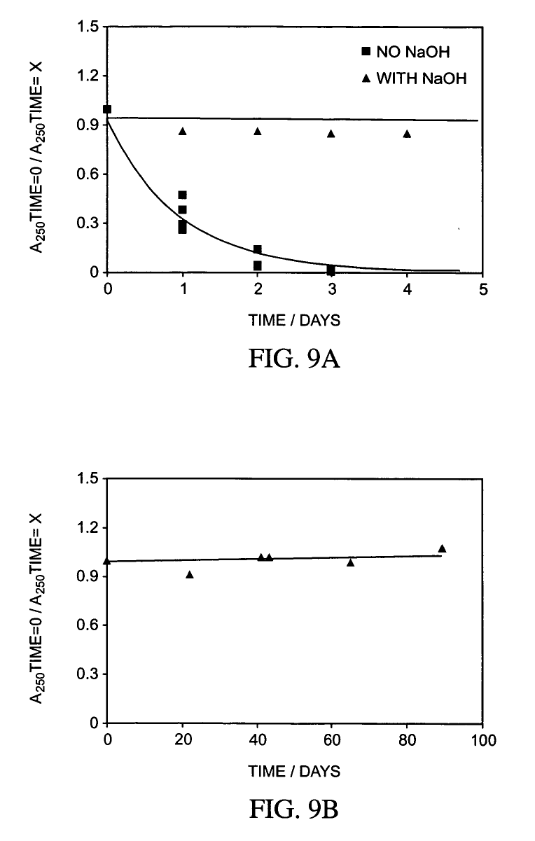

Three-dimensional supramolecular structures can be formed using functionalized nanoparticles formed by the methods disclosed herein using solution phase, and/or a combination of solution- and solid-phase synthesis. Multi-functionalized nanoparticles can be employed as building blocks to assemble complex supramolecular structures by applying sequential chemical methods. For example, as shown in FIG. 8D, subassemblies comprising more than one nanoparticle can be coupled to an immobilized subassembly. In certain embodiments, a subassembly can comprise a single nanoparticle, or more than one nanoparticle. Individual nanoparticles of a subassembly comprising more than one nanoparticle can be coupled to adjacent nanoparticles by covalent and/or non-covalent forces, and in certain embodiments can be covalently coupled by a ligand. Nanoparticles of a nanoparticle subassembly can comprise positionally distinguishable surface areas. FIGS. 9A-9D show the assembly of a nanoparticle structure comprising 16 nanoparticles formed by coupling two subassemblies, each comprising 8 nanoparticles. A first subassembly comprising 8 nanoparticles is immobilized on a functionalized SPS support surface (FIG. 9A), followed by coupling of a second subassembly comprising 8 nanoparticles to the immobilized first subassembly to form a nanoparticle structure immobilized on the functionalized support surface (FIGS. 9B-9C). A subassembly can comprise the same or different nanoparticles, and a first subassembly can comprise the same or different nanoparticles than a second subassembly. Additional materials such as additional nanoparticle subassemblies, nanoparticles, and/or ligands can be coupled to an immobilized nanoparticle subassembly or immobilized nanoparticle structure. The nanoparticle structure can then be released from the SPS support surface to form a free-standing 16-nanoparticle structure (FIG. 9D). In certain embodiments, a nanoparticle structure can be released from an SPS support surface by cleaving a photolabile linkage. Additional materials such as additional nanoparticle subassemblies, nanoparticles, and/or ligands can be coupled to a released nanoparticle subassembly or released nanoparticle structure. Adjacent nanoparticles of a nanoparticle structure can be coupled by covalent and/or non-covalent forces and in certain embodiments can be covalently coupled by ligands. Nanoparticles of a nanoparticle structure can comprise positionally distinguishable surface areas. Controlled assembly of a nanoparticle structure can be achieved by using multiple functional groups with different functionalities, and which can be selectively and independently removed or deactivated using appropriate chemistries.

### EXAMPLES

The invention is further illustrated by the following examples.

### Example 1

### SPS Modification of Au Nanoparticles Using Photolytic Decoupling General Methods

Toluene was purchased from J. T. Baker. Fmoc-Photolabile resin and Cysteine(Fmoc-Cys(Trt)-OH) were purchased from Advanced ChemTech. All other chemicals were purchased from Sigma-Aldrich. The DMF (N,N-Dimethylformamide) used was AldraSORB TM 99.8% with a water content less than 0.0005%. UV-Vis spectra were recorded on a Jassco V-550 spectrometer. Photochemical reactions were carried out using a UVP Inc. long wave ultraviolet lamp model B100-AP operating at 350 nm with a power output of 8,900 μW cm⁻² at 10 inches and 21,000 μW cm⁻² at 2 inches from the source. Transmission electron microscope data was recorded on a JEOL-2010 TEM operating at 200 KeV with carbon film coated copper grids.

Preparation of Gold Nanoparticles

Tetroctylammonium bromide (1.74 g) was dissolved in toluene (80 mL) to produce a 50 nM solution. This was combined with a 30 nM solution of hydrogen tetrachloroaurate (0.33 g) in distilled water (30 mL). The suspension was shaken until the tetrachloroaurate transferred to the organic phase. A fresh 0.4 M solution of sodium borohydride (0.40 g) in distilled water (25 mL) was prepared and added over 15 minutes with rapid stirring to the toluene and water mixture. On addition, the toluene phase changed color from a clear orange to an opaque deep ruby red. The aqueous phase remained colorless. The mixture was stirred for an additional 30 minutes before removing the lower aqueous layer. After separation, the toluene phase was washed twice with 0.5 M H₂SO₄ and diluted to 250 mL with fresh toluene. To improve the long-term stability of the Au-NP solution, the solution was stored over 0.1 M NaOH_{(aq)}.

The UV-Vis spectrum of the solution was recorded from 300 nm to 900 nm and showed the characteristic nanoparticle surface plasmon resonance peak at wavelength 520 nm. TEM image analysis showed discrete particles with an average diameter of 3.7 nm ±0.31 nm.

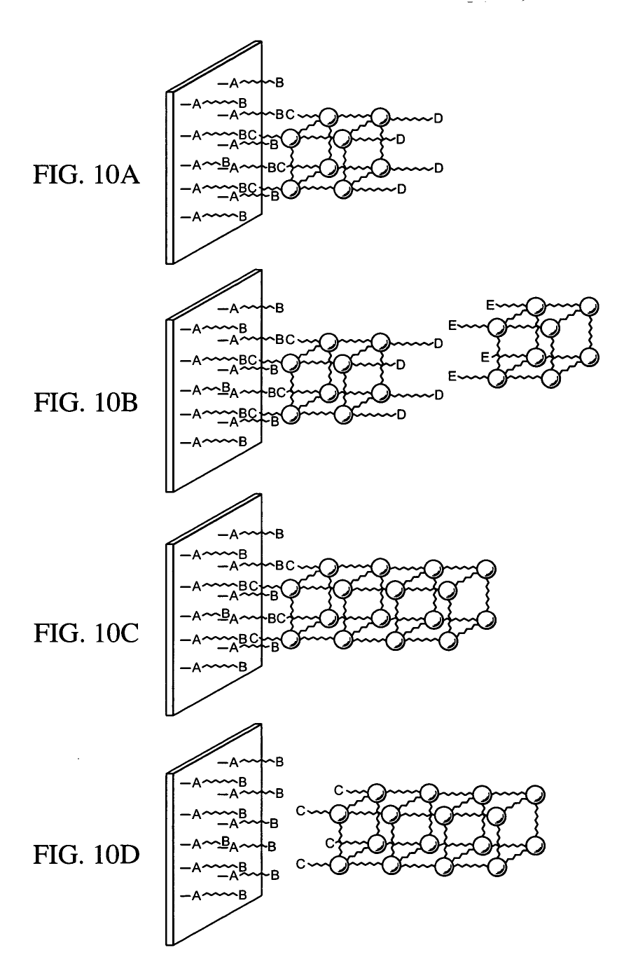

The long-term stability of Au nanoparticles synthesized using the foregoing method was determined by monitoring the intensity of the SPR peak over time. As shown in FIG. 1A, the intensity of the SPR peak corresponding to isolated Au nanoparticles diminished over a period of about 3 days for a solution of Au nanoparticles in toluene without NaOH. In contrast, Au-nanoparticles stored in toluene comprising 0.1M NaOH_{(aq)} resulted in the formation of a solution that was stable through at least 3 months (FIG. 10B).

Preparation of Fmoc-Cys(Trt) Loaded Photolabile Resin

Photolabile resin (SA5078 Advanced Chemtech) was first activated by removal of the Fmoc protecting group. The resin (18.5 mg) was suspended in a solution of 20% Piperidine and 80% DMF (2 mL) with agitation for approximately 30 min. A brown glass sample vial was used to minimize photoinduced reactions. After 30 minutes the solution was removed and the resin washed three times with fresh Piperidine/DMF solutions. The UV-Vis spectrum was observed to monitor the release of the Fmoc group.

Once activated, the Fmoc-Cys(Trt) amino acid was coupled to the resin following standard procedures using PyBOP. Fmoc-Cys(Trt)-OH (0.29 g) and PyBOP (0.26 g) were dissolved in DMF (2.6 mL), and DIPEA (170 μL) was added. The activated resin (18.5 mg) was then added and reacted with mild agitation for 24 hours at room temperature. Following completion of the coupling reaction, the resin was washed three times with fresh DMF, three times with ethanol, and dried to constant mass under vacuum. The extent of reaction was determined by the measuring the mass change of the resin and assaying the Trt content. There was no observable color change of the reaction mixture during the amino acid coupling reaction. The average percentage yield based on mass change for three activation reactions was 87% (73%, 96%, 93%).

Thiol Deprotection and TRT Assay

Initially the Cys thiol is protected with an acid labile trityl group. To activate the thiol group for reaction with a Au nanoparticle, the trityl group is removed. The Trt group on the Cys thiol was removed by washing the activated resin with 20% TFA/DCM (Trifluroacetic acid/Dichloromethane). The strong UV-Vis absorption of the Trt⁺ cation at 410 nm was used to estimate the amount of Cys coupled to the resin and to calculate a second percentage yield for comparison with the percentage yield based on mass change. The Fmoc-Cys(Trt) loaded photolabile resin (8 mg) was suspended in 20% TFA 80% DCM solution (1 mL) for 20 minutes. After this time the solution was removed and stored in a sealed container. This was repeated until the TFA/DCM solution no longer turned yellow on exposure to the resin. The TFA/DCM washings were then combined and diluted to 10 mL with fresh TFA/DCM. This solution was then further diluted (50 μL to 1 mL) in order to obtain the UV-Vis spectrum. The absorbance of the solution at 410 nm was 0.94, which provided an estimated ligand loading of 0.6 mmol-g⁻¹. The presence of the thiol group was further confirmed by a positive Ellman test (see, for example, Baydal, J. P., et al., Tetrahedron Letters, 2001, 42, 8531-8533).

Au Nanoparticle Binding

The photolabile resin (20 mg) loaded with Cys was placed in the Au nanoparticle solution (2.5 mL) and left for 24 hours. After this time the resin was removed and washed with a solution of dodecanethiol (3%) in toluene (97%) to remove Au nanoparticles not coupled to the resin via a thiol bond. This washing was repeated until no color was observed in the decanted solvent after mild sonication. The resin was then washed four times with fresh, clean toluene and dried to constant mass under vacuum. The mass increase associated with the binding of Au nanoparticle to the resin was between 3% and 6% with respect to the initial mass of the resin. The color of the resin changed from a light brown/yellow to a deep red/purple similar to the color of the Au nanoparticle solution.

Photochemical Decoupling

The photochemical decoupling reaction was carried out in toluene. Au nanoparticle coupled resin (20 mg) was suspended in toluene (10 mL) with stirring. The mixture was then exposed to UV light (λ=350 nm, intensity=8,900 μW cm⁻²) for 5 hours. After this time, the UV-Vis spectrum of the solution was recorded. The resin was removed, washed with fresh toluene and dried to constant mass under vacuum. The mass of the resin returned to within 10% of the initial mass of the resin prior to Cys loading and Au nanoparticle binding. The color of the resin changed from a deep red/purple to the initial brown/yellow color.

While certain embodiments have been shown and described, various modifications and substitutions may be made thereto without departing from the spirit and scope of the invention. Accordingly, it is to be understood that the present invention has been described by way of illustration and not limitations.

What is claimed is:

1. A method of forming a nanoparticle surface comprising a second ligand coupled to the nanoparticle surface comprising:
contacting the nanoparticle surface with a functionalized support surface comprising a first ligand;
coupling the first ligand to the nanoparticle surface; and
releasing the nanoparticle surface from the functionalized support surface to form a modified nanoparticle surface comprising the second ligand.

2. The method of claim 1, further comprising functionalizing a support surface with a plurality of first ligands to provide the functionalized support surface.

3. The method of claim 1, wherein the nanoparticle surface is stabilized.

4. The method of claim 1, wherein the functionalized support surface comprises a plurality of first ligands, wherein the plurality of first ligands disposed on the functionalized support surface is configured to couple a single first ligand to the nanoparticle surface.

5. The method of claim 1, wherein the functionalized support surface comprises a plurality of first ligands, wherein the plurality of first ligands disposed on the functionalized support surface is configured to couple a plurality of first ligands to the nanoparticle surface.

6. The method of claim 1, wherein the nanoparticle surface comprises a surfactant.

7. The method of claim 1, wherein the nanoparticle surface comprises a third ligand.

8. The method of claim 7, wherein the third ligand is reactive with the first ligand.

9. The method of claim 1, wherein of the first ligand comprises a photolabile linkage.

10. The method of claim 7, wherein the third ligand comprises a photolabile linkage.

11. The method of any one of claims 9 and 10, wherein the photolabile linkage is chosen from 2-Bromopropiophenone groups, o-Nitrobenzyl groups, and alkoxy substituted o-nitrobenzyl groups.

12. The method of claim 1, wherein releasing the nanoparticle surface comprises photolytically cleaving the first ligand.

13. The method of claim 1, wherein the modified nanoparticle surface comprises a positionally distinguishable surface area comprising the second ligand.

14. The method of claim 1, wherein the modified nanoparticle surface comprises a plurality of second ligands.

15. The method of claim 1, wherein the modified nanoparticle surface comprises a single second ligand.

16. The method of claim 1, wherein the second ligand comprises at least a portion of the first ligand.

17. The method of claim 1, further comprising coupling the second ligand to the nanoparticle surface while the nanoparticle surface is coupled to the functionalized support surface.

18. The method of claim 1, wherein a solution comprising the second ligand is contacted with the nanoparticle surface while the nanoparticle surface is coupled to the functionalized support surface.

19. A method of releasing a nanoparticle coupled to a surface comprising photolytically cleaving a photolabile linkage.

20. The method of claim 19, wherein the nanoparticle is coupled to the surface by a ligand comprising a photolabile linkage.

21. The method of claim 19, wherein the photolabile linkage is chosen from 2-Bromopropiophenone groups, o-Nitrobenzyl groups, and alkoxy substituted o-Nitrobenzyl groups.

22. A nanoparticle comprising a surface formed by the method of claim 1.

23. The nanoparticle of claim 22, comprising a single second ligand.

24. The nanoparticle of claim 22, comprising a plurality of second ligands.

25. The nanoparticle of claim 22, wherein each of the plurality of second ligands is independently chosen from the same ligands and different ligands.

26. The nanoparticle of claim 22, comprising a positionally distinguishable surface area, wherein the positionally distinguishable area comprises a second ligand.

27. A nanoparticle comprising a surface comprising a positionally distinguishable surface area, wherein the positionally distinguishable area comprises a ligand.

28. The nanoparticle of claim 27, comprising more than one positionally distinguishable area.

29. The nanoparticle of claim 27, wherein each area independently comprises a plurality of ligands chosen from the same ligands and different ligands.

30. The nanoparticle of claim 27, wherein each area independently comprises a single ligand chosen from the same ligand and a different ligand.

31. The nanoparticle of claim 27, comprising two positionally distinguishable surface areas.

32. A method of assembling a nanoparticle structure, comprising:
providing a functionalized support surface and a first subassembly;
immobilizing the first subassembly to the functionalized support surface; and
coupling a second subassembly to the first subassembly to form a nanoparticle structure immobilized on the functionalized support surface,
wherein the first and second subassemblies comprise a nanoparticle chosen from the same nanoparticle and a different nanoparticle.

33. The method of claim 32, further comprising coupling an additional subassembly to the immobilized nanoparticle structure, wherein the additional subassembly comprises a nanoparticle.

34. The method of claim 32, further comprising coupling a ligand to the immobilized nanoparticle structure.

35. The method of claim 32, further comprising releasing the nanoparticle structure from the support surface.

36. The method of claim 35, further comprising coupling a nanoparticle to the released nanoparticle structure.

37. The method of claim 35, further comprising coupling an additional subassembly to the released nanoparticle structure, wherein the additional subassembly comprises a nanoparticle.

38. The method of claim 32, wherein the nanoparticle structure comprises a chain of nanoparticles.

39. The method of claim 32, wherein the first and second subassemblies are independently chosen from a subassembly comprising a single nanoparticle and a subassembly comprising more than one nanoparticle.

40. The method of claim 32, wherein the nanoparticle comprises a positionally distinguishable surface area.

41. The method of claim 32, wherein releasing the nanoparticle structure comprises photolytically cleaving a photolabile linkage.

42. A nanoparticle structure formed by the method of claim 32.

43. A nanoparticle structure comprising a plurality of nanoparticles, wherein at least one of the plurality of nanoparticles comprises a positionally distinguishable surface area comprising a ligand.

44. The nanoparticle structure of claim 43, wherein adjacent nanoparticles are coupled by a ligand.
